(19) United States
(12) Patent Application Publication
(10) Pub. No.: US 2023/0255321 A1
(43) Pub. Date: Aug. 17, 2023

(54) Device and System for Measuring a Dimension of an Object

(71) Applicants: University of Louisville Research Foundation, Inc., Louisville (KY); Style Atlas Holdings Pty Ltd, Balmain (AU)

(72) Inventors: Peter Anthony Smit, Balmain (AU); Cindy Harnett, Louisville, KY (US)

(21) Appl. No.: 18/017,517

(22) PCT Filed: Jul. 23, 2021
(86) PCT No.: PCT/AU2021/050802

§ 371 Date: Jan. 23, 2023

Related U.S. Application Data
(60) Provisional application No. 63/056,387, filed on Jul. 24, 2020.

(51) Int. Cl. A43D 1/02; A61B 5/107

(52) U.S. Cl. CPC A43D 1/025; A61B 5/1079; A61B 5/1074; A61B 2503/12

(57) ABSTRACT

Provided is a device for measuring at least one dimension of an object. The device includes at least one body configured to be arranged along, or around, the object. The, or each, body carries an elongate stretchable waveguide and is configured to allow stretching the waveguide along its length when arranged along, or around, the object. The, or each, waveguide is associated with a sensor. The, or each, sensor includes a light emitter arranged and operable to emit at least one light pulse through the associated waveguide, and a light detector arranged to receive the at least one light pulse conveyed through the waveguide. The device also includes a communication module communicatively coupled with the, or each, sensor, and configured to communicate measurement data from the, or each, sensor to a processor to allow determining the at least one dimension. Systems and methods for measuring at least one dimension of an object are also disclosed.

### CROSS-REFERENCE TO RELATED APPLICATIONS

This application is the U.S. national phase of International Application No. PCT/AU2021/050802 filed Jul. 23, 2021, and claims priority to United States Provisional Patent Application No. 63/056,387, filed Jul. 24, 2020, the disclosures of which are hereby incorporated by reference in their entireties.

### BACKGROUND OF THE INVENTION

### Field of the Invention

The present disclosure relates, generally, to length measuring devices and, particularly, to a device for measuring an absolute length dimension defined by an object.

### Description of Related Art

Measuring an absolute length dimension defined by an object generally involves positioning a measuring device alongside, against, or around the object to allow recording the relevant dimension marked or displayed on the device. Devices such as rulers, calipers, or laser range finders are often used to measure linear distances, whereas flexible tape measures are typically used to measure non-linear distances, such as circumferential lengths.

Accurately measuring dimensions defined by a person's body can be difficult due to arranging a measure on or about the complex geometry defined by a body. Furthermore, the shape and/or dimensions defined by a body can be non-static due to the body being affected by the environment and/or parts of the body moving relative to each other. However, determining accurate dimensions of the body is required to understand if a garment will fit the body, particularly in instances where a garment is purchased without the purchaser being able to try the garment on to assess fit. Measuring the body is typically achieved by using a tape measure to measure various dimensions, such as the circumference of a waist, chest or head, or length of a leg or foot.

Obtaining accurate body dimensions allows selecting clothing which will fit according to a user's preference and particular body shape. However, even when the body is measured accurately, sizing of industrially manufactured clothing is often inconsistent. Sizing is variable as a generalised category, such as “size 10” or “XL” can relate to multiple garment dimensions. Some or all of the individual dimensions of different garments of the same size (e.g. size 10) can be significantly different. Even though garment sizing is guided by various standards, such as ISO 8559-1 to 8559-3 “Size designation of clothes”, the dimensions of a garment labelled as size “X” produced by one manufacturer can be significantly different to the dimensions of the same type of garment labelled as the same size produced by another manufacturer. Even the same size and type of garments from different clothing brands owned by the same parent company are known to have significantly different dimensions to each other. This is a result of a clothing brand basing the dimensions of their base size (or sample size) of each garment on a particular body, known as a fit model. Each brand's fit model will generally be different to another brand's fit model. Also, the sample size (e.g. size 10) of the fit model could be different for each brand. Each brand also decides on the difference between sizes for each of the key dimensions of each garment, known as grade rules. Thus two distinct brands that use the same fit model with the same sample size will have differently sized garments across their size range if they use different grade rules.

Online shopping for clothing is increasingly popular. Online retail websites or apps allow a garment size to be selected by a user from a range of available sizes. This may involve displaying a size guide for a category of garments, which is defined by the manufacturer. Purchased garments are typically delivered by post to the user. The user may find that the garment does not fit satisfactorily and then return the garment, typically by post, to the retailer for an exchange for a different size, or a refund. The inconvenience of this scenario, and the likelihood of it occurring, results in many potential users being hesitant to use online shopping for apparel and footwear purchases. To counteract this, many retailers offer to pay for the shipping of a purchase and also the return postage to compensate the user for the potential inconvenience of returning an item. This arrangement is highly inefficient for both user and retailer, is detrimental to the environment, and results in substantial costs for the retailer. Garment sizing inconsistencies between different manufacturers is one of, if not the, most common cause of returns of garments purchased online.

Any discussion of documents, acts, materials, devices, articles or the like which has been included in the present specification is not to be taken as an admission that any or all of these matters were common general knowledge in the field relevant to the present disclosure as it existed before the priority date of each of the appended claims.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

According to one disclosed aspect, there is provided a device for measuring at least one dimension of an object. The device includes at least one body configured to be arranged along, or around, the object. The, or each, body carries an elongate stretchable waveguide and is configured to allow stretching the waveguide along its length when arranged along, or around, the object. The, or each, waveguide is associated with a sensor. The, or each, sensor includes a light emitter arranged and operable to emit at least one light pulse through the associated waveguide, and a light detector arranged to receive the at least one light pulse conveyed through the waveguide. The device also includes a communication module communicatively coupled with the, or each, sensor, and configured to communicate measurement data from the, or each, sensor to a processor to allow determining the at least one dimension.

The, or each, sensor may be operable to measure a time period between emitting and receiving a light pulse, and the communication module be configured to communicate measured time period data received from the, or each, sensor to the processor.

The, or each, waveguide, may be configured as an elongate stretchable fiber and arranged to extend at least partway along the associated body.

The, or each, waveguide may be arranged in a loop to extend partway along the associated body in a first direction and partway along the body in a second, opposed direction, such that the loop defines a pair of parallel portions.

The device may be configured to measure at least one dimension of a limb of a user, and the, or each, body be configured as an elongate strap securable relative to the limb to extend along, or around, the limb.

The device may include a plurality of the straps and respective associated waveguides and sensors, and the straps be arranged such that securing each strap relative to the limb allows the communication module to communicate the measurement data from each sensor to the processor to cause the processor to determine a plurality of dimensions of the limb.

At least one strap may include an extendable portion and an inextensible portion, and the associated waveguide be arranged to extend at least partway along the extendable portion.

The device may include a spine, and the straps be arranged to be spaced along the spine, and each strap extend away from the spine.

The spine may define a longitudinal axis, and at least one of the straps be arranged to extend transversely to the axis, and at least one of the straps be arranged to extend parallel to the axis.

The device may be configured to receive a foot of a user, and the straps be dimensioned such that fitting the device to the foot causes each strap to be deformed by the foot to stretch each waveguide.

The straps may be arranged to allow operating the sensors to cause the processor to determine at least two of the following dimensions defined by the foot: foot length; ball girth; instep girth; and heel girth.

The, or each, waveguide may be manipulable to allow defining a non-linear path for the at least one light pulse.

According to another disclosed aspect, there is provided a system for determining at least one dimension defined by an object. The system includes the device according to any of the above paragraphs, and a processor configured such that, responsive to receiving measured data from the communication module of the device, the processor determines a length of the, or each, waveguide of the device, and, responsive to determining the length of the, or each, waveguide, the processor is further configured to determine the at least one dimension of the object.

According to a further disclosed aspect, there is provided a system for determining at least one dimension defined by an object. The system includes the device according to any of the above paragraphs, and a processor configured such that, responsive to receiving time period data measured by the, or each, sensor and communicated by the communication module, the processor determines a length of the, or each, waveguide, and, responsive to determining the length of the, or each, waveguide, the processor determines the at least one dimension of the object.

The, or each sensor, of the system described in the above paragraph may be operable to emit a plurality of light pulses and measure a complementary plurality of time periods to cause the measured time periods to be communicated to the processor, and the processor be configured to average a defined plurality of the received measured time periods, and determine the length of the, or each, waveguide based on the averaged measured time period.

The processor of any of the above described systems may be hosted remotely from the device, and the communication module be configured to wirelessly communicate with the processor.

According to yet another disclosed aspect, there is provided a system for determining at least one dimension defined by an object. The system includes the device according to any of the above paragraphs configured to measure at least one dimension of a limb of a user, and a processor configured such that, responsive to receiving the measured data from the communication module of the device, the processor determines a length of the, or each, waveguide, and, responsive to determining the length of the, or each, waveguide, the processor determines the at least one dimension of the limb of the user, the processor being further configured such that, responsive to determining the at least one dimension, the processor determines a garment size corresponding with the at least one dimension.

The system may be configured such that responsive to the processor receiving the measured data from the communication module of the device, the processor is configured to determine a plurality of dimensions of the foot of the user, and, responsive to determining the plurality of dimensions, determine a shoe size corresponding with the plurality of dimensions.

According to a further disclosed aspect, there is provided a method for measuring at least one dimension of an object. The method includes: arranging an elongate stretchable waveguide along the object such that the waveguide is stretched, the waveguide associated with a sensor including a light emitter arranged and operable to emit at least one light pulse through the waveguide, and a light detector arranged to receive the at least one light pulse conveyed through the waveguide; operating the sensor to cause a light pulse to be emitted by the emitter, travel through the waveguide, and be received by the detector, to generate measurement data; and communicating the measurement data to a processor, causing the processor to determine the at least one dimension.

It will be appreciated embodiments may comprise steps, features and/or integers disclosed herein or indicated in the specification of this application individually or collectively, and any and all combinations of two or more of said steps or features.

### BRIEF DESCRIPTION OF THE DRAWINGS

Embodiments will now be described by way of example only with reference to the accompany drawings in which:

FIG. 1 is a plan view of a device for measuring at least one dimension defined by an object, the device adapted to measure four dimensions defined by a foot of a user and in a pre-assembly configuration to form a pattern;

FIGS. 2 and 3 are perspective views of the device shown in the FIG. 1 in an assembled configuration to form a sock for receiving the foot;

FIG. 4 is a perspective view of the device shown in FIGS. 2 and 3 fitted to the user's foot;

FIGS. 5 to 7 are plan and side view diagrams illustrating dimensions of the foot which are measurable by operating the device shown in FIGS. 2 to 4; and

FIG. 8 includes a plot indicating length measurements obtained using three different waveguides, a schematic of a strap carrying a waveguide, and a schematic of a light sensor arranged relative to ends of a looped waveguide.

### DESCRIPTION OF THE INVENTION

In the drawings, reference numeral 10 generally designates a device 10 for measuring at least one dimension of an object. The device 10 includes at least one body 14 configured to be arranged along, or around, the object. The, or each, body 14 carries an elongate stretchable waveguide 16 and is configured to allow stretching the waveguide 16 along its length when arranged along, or around, the object. The, or each, waveguide 16 is associated with a sensor 22. The, or each, sensor 22 includes a light emitter 24 arranged and operable to emit at least one light pulse through the associated waveguide 14, and a light detector 26 arranged to receive the at least one light pulse conveyed through the waveguide 16. The device 10 also includes a communication module 28 communicatively coupled with the, or each, sensor 22, and configured to communicate measurement data from the, or each, sensor 22 to a processor 30 to allow determining the at least one dimension.

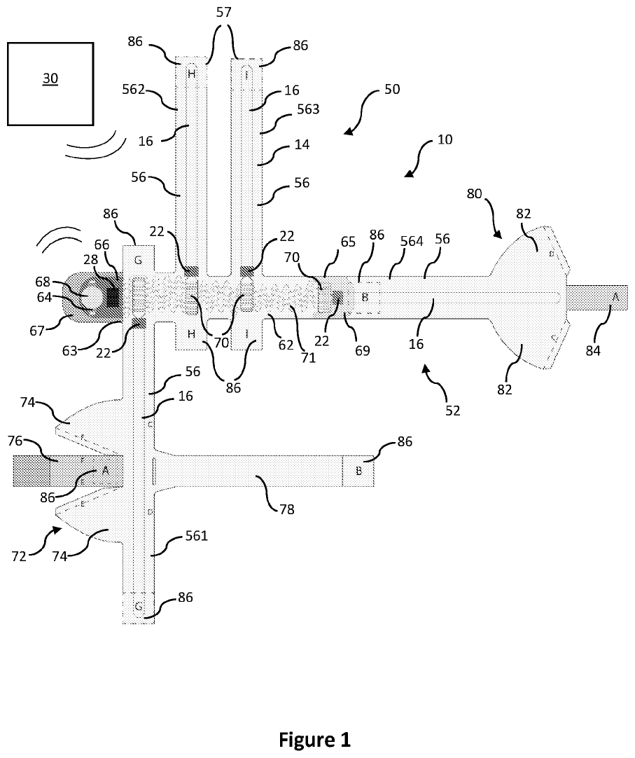

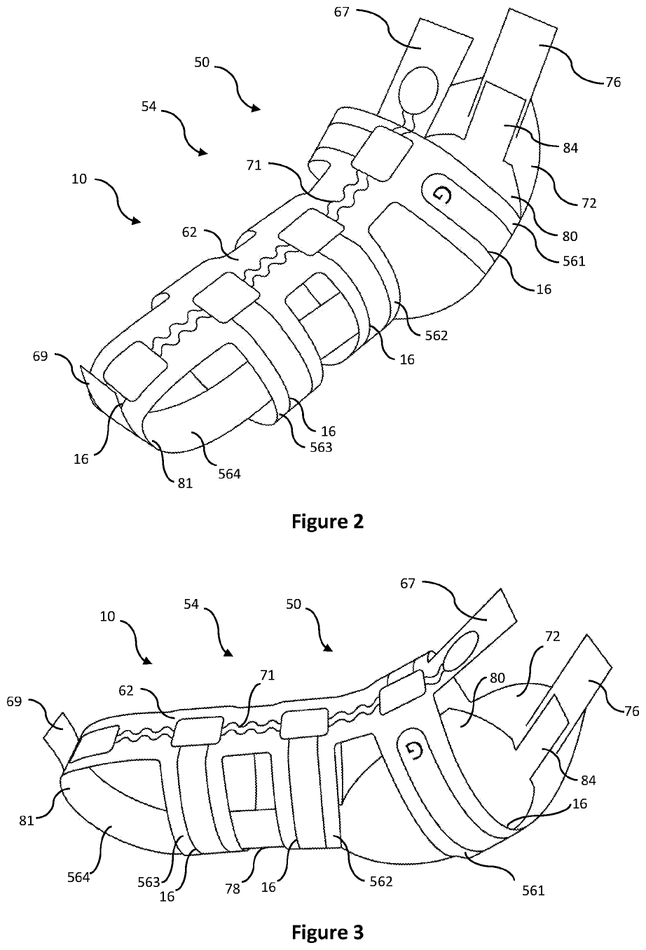

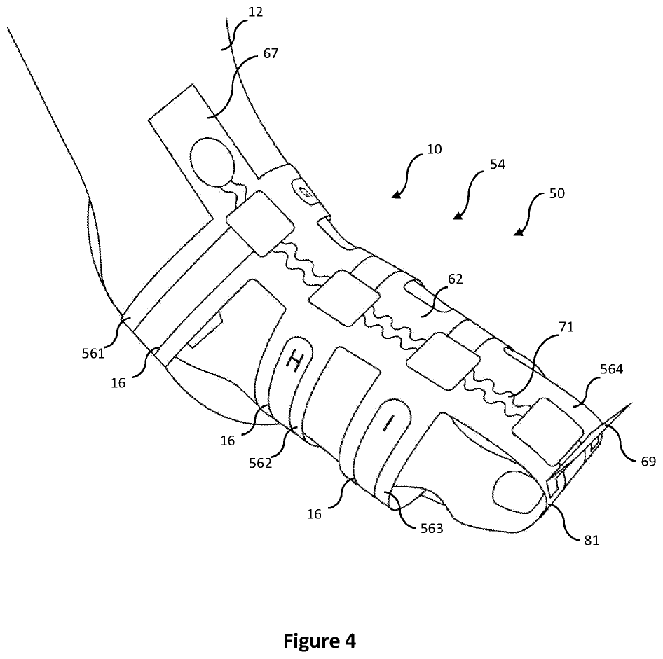

FIGS. 1 to 4 show an embodiment 50 of the device 10 configured to allow measuring a plurality of dimensions of a user's limb, and particularly dimensions defined by a user's foot 12. FIG. 1 illustrates the device 50 configured in a pre-assembly state as a flat pattern 52. FIGS. 2 and 3 illustrate the device 50 configured in an assembled state to define a sock 54 shaped to receive the foot 12. FIG. 4 shows the device 50 fitted to a user's foot 12 during use. It will be appreciated that the device 50 shown in FIGS. 1 to 4 is configured to receive a defined range of foot sizes and that the device 50 may be scaled up or down in size and proportions to allow receiving a different range of foot sizes. It will also be appreciated that the device 10 is alternatively configurable to allow measuring other limbs or regions of a user's body, such as being configured as a head band, a glove, a shirt, a compression vest, pants, leggings or tights.

In the illustrated embodiment 50, the at least one body 14 is in the form of a plurality of flexible straps 56 arranged to allow being secured along, including about, a plurality of regions of the foot 12. Each strap 56 carries a waveguide 16 connected via fixtures at each end 18, 20 to a sensor 22 to define a closed path for the light pulse. The strap 56 is typically formed from a stretchable fabric and includes an inextensible portion or tab 57 arranged at least adjacent the sensor 22, and typically at each end of the strap 56, to prevent mechanical strain being transmitted to the sensor 22 housing during use. Each strap 56 may comprise two layers of the stretchable fabric arranged to sandwich the waveguide 16, the layers being bonded to each other, such as with a heat-bondable stretchable film.

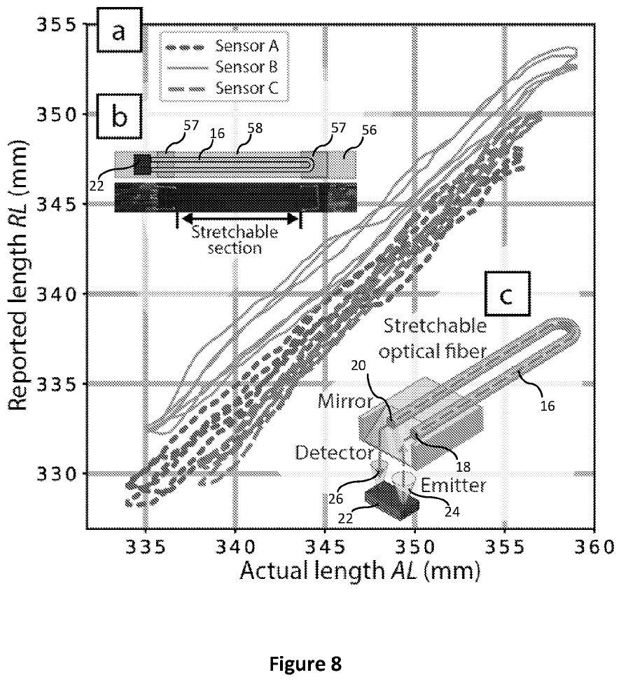

A schematic of one of the straps 56 and associated waveguide 16 is shown in FIG. 8, part (b), and a schematic of one of the sensors 22 and associated waveguide 16 is shown in FIG. 8, part (c). It will be appreciated that the mirror shown in FIG. 8 may be absent from the device 50, for example, by arranging the sensor 22 to be aligned with the waveguide 16. FIG. 8 part (a) shows data obtained from three fabric-embedded stretchable optical fiber waveguides 16 and associated sensors 22, the plots generated from three cycles of strain exerted on the waveguides 16 and showing known length after a ten-point moving average was performed on the as-received length data, as discussed in greater detail below.

In the illustrated embodiment 50, each waveguide 16 is fixed to the strap 56 via stitched thread. In some embodiments, at least one of the waveguides 16 is bonded to the associated strap 56 via a stretchable adhesive, such as carried by a stretchable polymer sheet. It will be appreciated that the number of straps 56 and associated waveguides 16 and sensors 22 included in the device 50 is exemplary and that the device 10 is alternatively configurable to include more or fewer straps 56 and associated waveguides 16 and sensors 22 depending on usage requirements. For example, in embodiments where the device 10 is configured as a headband (not illustrated), the headband is effectively configured as a single strap carrying a single waveguide 16 and sensor 22.

Best shown in FIGS. 2 and 3, in the assembled configuration, each strap 56 is fixed at each end to define a loop to allow receiving a region of the foot 12. In other embodiments (not illustrated), at least one strap 56 defines a free end and is configured to be releasably securable about the limb. In some embodiments, this arrangement allows adjusting the length of the strap 56.

Each sensor 22 is operable to emit pulses of light at a defined frequency, typically being around 30 Hz. Each waveguide 16 is configured to convey electromagnetic waves, including visible light. Each sensor 22 is typically arranged adjacent the ends 18, 20 of the waveguide to cause a light pulse to be conveyed from one end 18 to the other 20. In some embodiments (not illustrated), the light emitter 24 is arranged at one location along the waveguide 16 and the light detector 26 is arranged at another, spaced location, such that the light pulse only travels through a portion of the waveguide 16. In the illustrated embodiment 50, each waveguide 16 is in the form of an elongate stretchable fiber, such as a stretchable optical fiber having an elastic response to strain of up to 100%. Such fibers comprise a core and a coating surrounding the core, the core having a refractive index which is greater than the refractive index of the coating. The fibers may comprise a core formed from polyurethane and a cladding formed from silicone. In such embodiments, the polyurethane core has a refractive index of n=1.53 and diameter of 1 mm, and the silicone cladding has a refractive index of n=1.41 and a thickness of 0.2 mm. Examples of such fibers are described in international patent application no. PCT/US2019/015520, the disclosure of which is incorporated herein in its entirety.

Best shown in FIG. 1, each waveguide 16 is arranged along the associated strap 56 in a loop, with the ends 18, 20 arranged adjacent to each other and connected to the associated sensor 22. In the illustrated embodiment 50, each waveguide 16 is arranged to extend substantially along the entire length of the strap 56, in two opposed directions, to form a pair of parallel portions joined by a U-bend portion. In other embodiments (not illustrated), the fiber 28 is arranged to extend only partway along the strap 56.

The arrangement of the waveguide 16 relative to the length of the strap 56 may be configured according to the dimension and/or geometry of the object which the strap 56 is intended to measure to allow measuring the length within an acceptable accuracy range. For example, where the dimension is a moderate length, such as 1 m, along a plane defined by the object, the waveguide 16 may be arranged as a singular bead along only a portion of the length of the strap 56, as this defines a length of waveguide 16 sufficient to allow accurately determining the dimension defined by the object. In other embodiments, where the dimension is a short length, such as 5 mm, around a shaft, the waveguide 16 may be arranged to extend back and forth a plurality of times, defining a plurality of U-turns, and substantially along the length of the strap 56, as this defines a length of waveguide 16 necessary to allow accurately determining the dimension defined by the object.

In some embodiments, at least one of the straps 56 includes an extendable portion 58 interposed between a pair of spaced inextensible portions 57, and the associated waveguide 16 is arranged to extend across the extendable portion 58 and between the inextensible portions 57. In such embodiments, each inextensible portion 57 defines a static length of the waveguide 16 which may be included as a constant, in addition to a measured length, when the processor 30 calculates length using measurements recorded with the device 50.

In the illustrated embodiment 50, each strap 56 extends from a spine 62 which defines opposed ends 63, 65 and a longitudinal axis between the ends 63, 65. The spine 62 is typically formed from the same material as the straps 56 and is resiliently deformable to allow conforming to contours of the foot 12. In some embodiments, the spine 62 and straps 56 are formed from a blend of nylon and elastane, which may be around 86% nylon and 16% elastane. The spine 62 carries various electrical components, including a PCB 64 carrying a low power microprocessor 66, the communication module 28 including an antenna, and a battery 68. The microprocessor 66 is typically configured to coordinate communicating measured data from the sensors 22 via the communication module 28 to the processor 30. The spine 62 also carries flexible interposers 70 connecting the sensors 22 to conductive thread 71 coupled to the battery 68 and PCB 64. The thread 71 allows supplying electrical power to the sensors 22, and communicating data to the PCB 64. The communication module 28 is typically configured for ultra-high frequency radio wave (wireless) communication, such as according to the Bluetooth standard.

In some embodiments, the spine 62 is configured to be flexible but substantially non-extendible between its opposed ends 63, 65 and each strap 56 has an inextensible portion 60 which is an integral part of, or joined to, the spine 62. In the illustrated embodiment 50, an inextensible first tab 67 and second tab 69 are arranged to extend away from a respective end 63, 65 of the spine 62. Best shown in FIG. 2, in the assembled configuration, the first tab 67 and second tab 69 extend away from the spine 62 to allow each tab 67, 69 to be manually grasped to assist fitting the device 50 to the foot 12.

The straps 56 are arranged to be spaced along the longitudinal axis of the spine 62 with at least one strap 56 extending transversely to the axis, and at least one strap 56 extending parallel to the axis. The first strap 561, second strap 562 and third straps 563 extend substantially perpendicularly to the axis, and a fourth strap 564 extends parallel to the axis from one end 65 of the spine 62. The arrangement of the straps 56 relative to the spine 62 in this way allows the straps 56 and associated waveguides 16 to be arranged around and/or along specific regions of the foot 12. It will be appreciated that, in other embodiments (not illustrated), more or less straps 56 may be arranged along the spine 62, and at different angles relative to the longitudinal axis, depending on the dimension(s) the device 50 is configured to measure.

A first heel cup portion 72 is arranged to extend from one side of the first strap 561. The heel cup portion 72 comprises a pair of wings 74 securable to an inextensible third tab 76. Best shown in FIGS. 2 and 3, when in the assembled configuration, the third tab 76 is arranged to extend away from the heel cup portion 72 to allow being manually grasped to assist fitting the device 50 to the foot 12, typically at the same time as the first tab 67, to allow enlarging the opening defined between the heel cup portion 72 and the first strap 561.

A sole plate 78 extends from the first strap 561 opposite to the first heel cup portion 72. Best shown in FIG. 3, when in the assembled configuration, the sole plate 78 defines a base for the device 50. In some embodiments (not illustrated), the sole plate 78 is shaped and dimensioned to substantially cover the sole of the user's foot 12. In such embodiments, the sole plate 78 may carry one or more embedded force sensors communicatively coupled with the communication module 28 and operable to measure force exerted on the sole plate 78 by the user when standing. This can allow the processor 30 to determine pressure points and/or postural issues for the user.

Best shown in FIG. 1, a second heel cup portion 80 is arranged adjacent to the end of the fourth strap 564. The second heel cup portion comprises a further pair of wings 82 securable to a fourth tab 84. Best shown in FIGS. 2 and 3, when in the assembled configuration, the fourth strap 564 is arranged against the sole plate 78 to form a toe receiving portion 81 and arrange the second heel cup portion 80 to be nested and fixed within the first heel cup portion 72.

Also best shown in FIG. 1, the device 50 includes a plurality of connecting portions 86 arranged to allow assembling the device 50 from the pre-assembled state (FIG. 1) to the assembled state (FIGS. 2 and 3). Each connecting portion 86 is identified by a pair of corresponding labels A-A, B-B, G-G, H-H, and I-I. Fixing the pairs of connecting portions together allows assembling the device 50 from the pre-assembled configuration to the assembled configuration. Each pair of connecting portions 86 is typically fixed together by stitched thread and/or bonding.

Best shown in FIGS. 2 and 3, when in the assembled configuration, the connecting portion 86A of the fourth tab 84 is joined to connecting portion 86A on the third tab 76, the connecting portion 86B of the fourth strap 564 is joined to the connecting portion 86B of the sole plate 76, the connecting portion 86G of the first strap 561 is joined to the connecting portion 86G extending from the spine 62, the connecting portion 86H of the second strap 562 is joined to the connecting portion 86H extending from the spine 62, and the connecting portion 861 of the third strap 563 is joined to the connecting portion 861 extending from the spine 62.

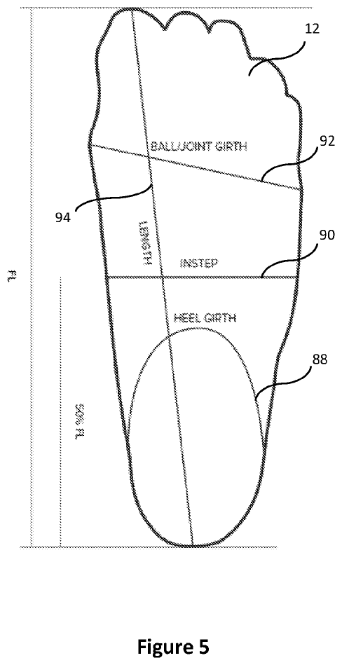

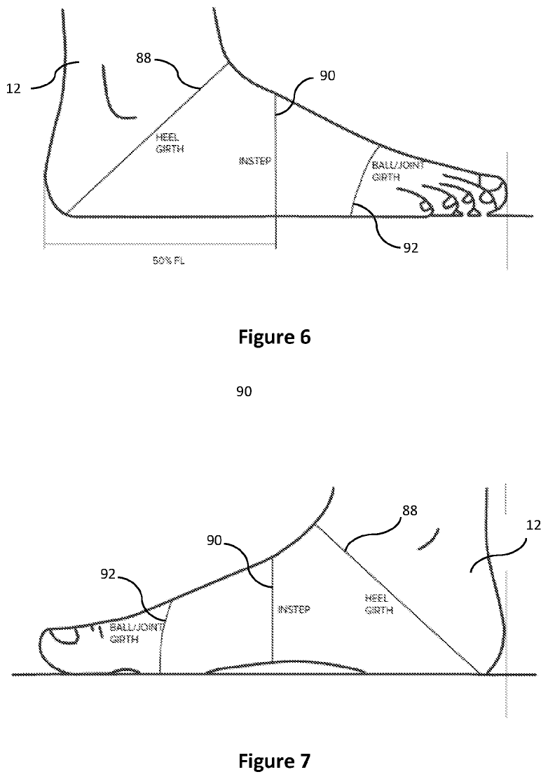

FIGS. 5 to 7 illustrate the dimensions which the device 50 is configured to allow measuring when in the assembled configuration and fitted to the foot 12 (FIG. 4). The first strap 561 is arranged to form a loop to encircle the user's heel to allow measuring heel girth 88, typically being the circumference of the foot between where the heel touches the floor and the junction between top of the foot 12 and leg. The second strap 562 is arranged to form a loop to encircle the user's instep, typically being midway along the length of the foot 12, to allow measuring instep girth 90. The third strap 563 is arranged to form a loop to encircle the ball of the user's foot 12 to allow measuring ball girth 92, typically being the circumference of the foot between the metatars-phalangeal joint on the big toe and the small toe. The fourth strap 564 is arranged to extend along the sole of the user's foot 12, at least partially between heel and big toe, to allow measuring foot length 94, typically being the maximum length of the base of the foot 12, between the heel and the longest toe (typically the big toe). It will be appreciated that each strap 56 is dimensioned to define a length which is less than the minimum foot size the device 50 is configured to measure, meaning that each strap 56 is stretched in length when the user's foot 12 is received in the device 50.

In the illustrated embodiment 50, each sensor 22 includes a microprocessor configured to determine a time period between emitting a light pulse, from the emitter 24, and receiving the emitted light pulse, by the detector 26—known as “time-of-flight”. The microprocessor is further configured to communicate time period measurement data to the communication module 28 to cause the measurements to be communicated to the processor 30. In some embodiments, each sensor 22 is configured as a miniature LiDAR sensor module, such as the VL53LOX laser ranging module manufactured by ST Microelectronics NV. This sensor module can be particularly compatible with urethane core fibers, as discussed above, as the integrated 940 nm wavelength laser light emitter corresponds with the transmission range of a urethane core. It will be appreciated that the light emitter 24 is operable to emit a range of other wavelengths to allow measuring time-of-flight, typically between 200 to 2,000 nm.

In other embodiments, the device 10 includes alternative sensors 22 configured to measure data according to one or more of: electronic or acoustic time domain reflectometry; optical interference; optical linear encoding; electronic resistive encoding; and capacitive linear encoding. It will be appreciated that operating such sensors 22 allows obtaining data which can be processed to determine length of the associated waveguide 16.

In some embodiments, the device 10 forms part of a system 100 also including the processor 30. In such embodiments, the processor 30 is configured such that, responsive to receiving measured data from the communication module 28, the processor 30 determines a length of the, or each, waveguide 16, and, responsive to determining the length of the, or each, waveguide 16, the processor determines the at least one dimension of the object.

In such embodiments, the, or each, sensor 22 may include a microprocessor configured to determine a time period between emitting a pulse, from the emitter 24, and receiving the emitted pulse, by the detector 26. The microprocessor is further configured to communicate time period measurement data to the communication module 28 to cause the measurements to be communicated to the processor 30. In such embodiments, the processor 30 may be further configured such that responsive to receipt of a defined plurality of time period measurements from the communication module 28, the processor 30 calculates the average of the plurality of time period measurements, and determines the length of the, or each, waveguide 16 based on the averaged measured time period, and consequently determines the at least one dimension of the object. For example, in such embodiments, the processor 30 may be configured such that response to receiving ten time period measurements, typically received in less than one second, the processor 30 calculates the average of the ten time periods and determines the length of the waveguide 16 based on the average.

Regardless of whether the processor 30 determines the length of the waveguide 16 based on a single received time period measurement, or an average of a defined plurality of measurements, the processor 30 is configured to calculate the length as the product of the measured time period, typically in the order of nanoseconds, and the speed of light through the waveguide 16.

The processor 30 is typically hosted remotely from the device 10 and configured to communicate wirelessly with the communication module 28. In some embodiments, the processor 30 is hosted in a personal computing device, such as a smartphone or tablet computer, and configured by an application (software) to determine length based on measurements recorded by the sensor(s) 22. In such embodiments, the application may be configured to cause the processor 30 to display a specific user interface (UI) to allow the user to operate the device 10 to cause measuring the at least one dimension. In other embodiments, the processor 30 is a component of the device 10, such as being carried on the PCB 64 of the device 50.

In some embodiments, the device 50 forms part of a system 110 also including the processor 30. In such embodiments, the processor 30 is configured such that, responsive to receiving the measured data from the communication module 28, the processor determines a length of at least one of the waveguides 16, and, responsive to determining the length of the at least one waveguide 16, the processor 30 determines a corresponding at least one dimension defined by the limb of the user. Typically, the processor 30 is configured to determine the length of each waveguide 16 and, as a result, determine a plurality of dimensions defined by the limb, including any of foot length, ball girth, instep girth, and heel girth.

The processor 30 may be further configured such that, responsive to determining the dimension(s) defined by the limb, the processor 30 determines a garment size corresponding with the dimension(s). For example, where the device 50 is operated to allow the processor 30 to determine one or more of, and typically all of, foot length, ball girth, instep girth, and heel girth, the processor 30 is configured to compare the determined foot measurements with a sizing data table, such as “last” measurements, defined by a shoe manufacturer, and select the appropriate shoe size to fit the user's foot 12, and/or deliver shoe fitting guidance to the user.

In some embodiments, the processor 30 is configured to determine the dimension(s) defined by the limb multiple times, such as during a defined period. This may allow determining limb measurements as the limb moves through a specific range of motion which affects the dimensions of the limb, such as moving between a relaxed position of the foot 12 and flexing the arch of the foot 12. Determining the range of measurements consequently allows the processor 30 to derive targeted shoe fitting guidance, for example, such as determining one or more stiletto shoes which would be suitable for the user's foot 12.

Use of the device 10 involves: arranging at least one waveguide 16 along the object such that the waveguide is stretched; operating the sensor 22 associated with the at least one waveguide 16 such that the light emitter 24 emits a pulse of light through one end of the waveguide 16, causing the pulse to travel through the waveguide 16 and be received by the detector 26 to generate measurement data; and communicating the measurement data to the processor 30 to cause the processor 30 to determine the at least one dimension defined by the object.

Arranging the waveguide 16 along the object typically involves wrapping the waveguide 16 at least partially along a peripheral region of the object. Additionally or alternatively, this may involve stretching the waveguide along a planar region or contour of an object. For example, in some embodiments, such as the device 50, the waveguide 16 is arranged to at least partially surround and be deformed by a region of the object. In other embodiments, the waveguide may be stretched linearly from one location defined by the object to another location defined by the object.

Use of the device 50 involves: the user gripping tab 67 and/or tab 76 and urging the foot 12 of the user through the opening defined between the heel cup portion 72 and the first strap 561 to be received within each of the first strap 561, second strap 562 and third strap 563, and to press against the toe receiving portion 81 defined by the fourth strap 564, causing each of the straps 56 and associated waveguides 16 to tension and stretch, as shown in FIG. 4; operating each sensor 22 that the light emitter 24 emits a pulse of light through one end of the associated waveguide 16, causing the pulse to travel through the waveguide 16 and be received by the detector 26 to generate measurement data; and communicating the measurement data to the processor 30 to cause the processor 30 to determine a plurality of dimensions defined by the foot 12.

The device 10 allows automating measuring one, or more, dimensions of an object by stretching a waveguide 16 around, or along, the object. This is readily achieved by a single, unskilled user. This approach typically produces highly accurate results comparable to significantly more complex, expensive, and less portable 3D scanners.

In some embodiments, the device 10 is operable to measure more than one dimension simultaneously, and/or measure a range of dimensions as the object is moved or deformed. This allows recording a greater scope of measurements than is achievable with conventional approaches.

In some embodiments, the device 10 is operable to measure the time period for a light pulse to be conveyed through the waveguide 16—known as “time-of-flight”. Such embodiments significantly simplify construction of the transmission path, which can be an optical waveguide, an acoustic waveguide, such as a hollow tube, or in the case of electronic time domain reflectometry, a wire. Furthermore, this approach can substantially enhance accuracy and reliability of determining length of the waveguide 16.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1-19. (canceled)

20. A device for measuring at least one length dimension of an object, the device comprising:
at least one body having at least a portion configured to be extendable and arranged along, or around, the object, the, or each, body carrying an elongate stretchable waveguide configured to elastically stretch along its length, the, or each, body connected to the associated waveguide to allow causing significant stretching of the waveguide along its length when the body and the waveguide are tensioned along, or around, the object;
the, or each, waveguide associated with a sensor, the, or each, sensor comprising a light emitter arranged and operable to emit at least one light pulse through the associated waveguide, and a light detector arranged to receive the at least one light pulse conveyed through the waveguide, the, or each, sensor operable to measure a time period between emitting and receiving a light pulse; and
a communication module communicatively coupled with the, or each, sensor, and configured to communicate measured time period data from the, or each, sensor to a processor to cause determining the at least one length dimension based on the measured time period data.

21. The device according to claim 20, wherein the, or each, waveguide, is configured as an elongate stretchable fiber and arranged to extend at least partway along the associated body.

22. The device according to claim 21, wherein the, or each, waveguide is arranged in a loop to extend partway along the associated body in a first direction and partway along the body in a second, opposed direction, such that the loop defines a pair of parallel portions.

23. The device according to claim 20, configured to measure at least one length dimension of a limb of a user, and wherein the, or each, body is configured as an elongate strap securable relative to the limb to extend along, or around, the limb.

24. The device according to claim 23, comprising a plurality of the straps and respective associated waveguides and sensors, the straps arranged such that securing each strap relative to the limb allows the communication module to communicate the measurement data from each sensor to the processor to cause the processor to determine a plurality of length dimensions of the limb.

25. The device according to claim 24, wherein at least one strap comprises an extendable portion and an inextensible portion, and wherein the associated waveguide is arranged to extend at least partway along the extendable portion.

26. The device according to claim 24, comprising a spine, and wherein the straps are arranged to be spaced along the spine, and each strap extend away from the spine.

27. The device according to claim 26, wherein the spine defines a longitudinal axis, and at least one of the straps is arranged to extend transversely to the axis, and at least one of the straps is arranged to extend parallel to the axis.

28. The device according to claim 24, configured to receive a foot of a user, and the straps are dimensioned such that fitting the device to the foot causes each strap to be deformed by the foot to stretch each waveguide.

29. The device according to claim 28, wherein the straps are arranged to allow operating the sensors to cause the processor to determine at least two of the following dimensions defined by the foot: foot length; ball girth; instep girth; and heel girth.

30. The device according to claim 20, wherein the, or each, waveguide is manipulable to allow defining a non-linear path for the at least one light pulse.

31. A system for determining at least one length dimension defined by an object, the system comprising:
the device according to claim 20; and
a processor configured such that, responsive to receiving measured time period data from the communication module, the processor determines a length of the, or each, waveguide, and, responsive to determining the length of the, or each, waveguide, the processor determines the at least one length dimension of the object.

32. The system according to claim 31, wherein the, or each sensor, is operable to emit a plurality of light pulses and measure a complementary plurality of time periods to cause the measured time periods to be communicated to the processor, and the processor is configured to average a defined plurality of the received measured time periods, and determine the length of the, or each, waveguide based on the averaged measured time period.

33. The system according to claim 31, wherein the processor is hosted remotely from the device, and the communication module is configured to wirelessly communicate with the processor.

34. A system for determining garment size for a user, the system comprising:
the device according to claim 23; and
a processor configured such that, responsive to receiving the measured time period data from the communication module, the processor determines a length of the, or each, waveguide, and, responsive to determining the length of the, or each, waveguide, the processor determines the at least one length dimension of the limb of the user,
the processor further configured such that, responsive to determining the at least one length dimension, the processor determines a garment size corresponding with the at least one length dimension.

35. A system for determining garment size for a user, the system comprising the device according to claim 28, and a processor configured such that, responsive to receiving the measured data from the communication module, the processor determines a length of the, or each, waveguide, and, responsive to determining the length of the, or each, waveguide, and, responsive to determining the length of the, or each, waveguide, the processor is configured to determine a plurality of dimensions of the foot of the user, and, responsive to determining the plurality of dimensions, determine a shoe size corresponding with the plurality of dimensions.

36. A method for measuring at least one length dimension of an object, the method comprising:
arranging an elongate elastically stretchable waveguide along the object such that the waveguide is substantially stretched along its length, the waveguide associated with a sensor including a light emitter arranged and operable to emit at least one light pulse through the waveguide, and a light detector arranged to receive the at least one light pulse conveyed through the waveguide, the sensor operable to measure a time period between emitting and receiving a light pulse;
operating the sensor to cause a light pulse to be emitted by the emitter, travel through the waveguide, and be received by the detector, to generate time period measurement data; and
communicating the time period measurement data to a processor, causing the processor to determine the at least one length dimension based on the time period measurement data.
